(19) United States
(12) Patent Application Publication
(10) Pub. No.: US 2016/0287532 A1
(43) Pub. Date: Oct. 6, 2016

(54) CURCUMIN FOR TREATING INTERVERTEBRAL DISC DISEASE

(71) Applicant: SignPath Pharma Inc., Quakertown, PA (US)

(72) Inventors: Diana C. Sordillo, New York, NY (US); Peter P. Sordillo, New York, NY (US); Lawrence Helson, Quakertown, PA (US)

(21) Appl. No.: 15/082,708

(22) Filed: Mar. 28, 2016

Related U.S. Application Data
(60) Provisional application No. 62/141,583, filed on Apr. 1, 2015.

(51) Int. Cl. A61K 31/12; A61K 49/00; A61K 45/06; A61K 9/127

(52) U.S. Cl. CPC A61K 31/12; A61K 9/127; A61K 49/0004; A61K 45/06

(57) ABSTRACT

The present invention includes a method of treating an intervertebral disease or condition comprising: identifying a patient in need of treatment for the intervertebral disease or condition; and administering to the patient an amount of an anti-inflammatory agent that causes a cardiac channelopathy, such as curcumin, and a liposome, wherein the liposome is provided in an amount sufficient to reduce or eliminate the cardiac channelopathy caused by the anti-inflammatory agent, and the anti-inflammatory agent is provided in an amount sufficient to treat or ameliorate the symptoms of the intervertebral disease or condition.

### CROSS-REFERENCE TO RELATED APPLICATIONS

This application claims priority to U.S. Provisional Application Ser. No. 62/141,583 filed Apr. 1, 2015, the entire contents of which are incorporated herein by reference.

### STATEMENT OF FEDERALLY FUNDED RESEARCH

None.

### INCORPORATION-BY-REFERENCE OF MATERIALS FILED ON COMPACT DISC

None.

### TECHNICAL FIELD OF THE INVENTION

The present invention relates in general to the field of pain management and treatment for intervertebral disc disease, and more particularly, to the use of curcumin or curcuminoids to treat symptoms of intervertebral disc disease.

### BACKGROUND OF THE INVENTION

Without limiting the scope of the invention, its background is described in connection with intervertebral disc disease.

Some degree of degeneration of the intervertebral disc (IVD) occurs in up to 97% of adults by age fifty (1). While intervertebral disc disease can be asymptomatic, intervertebral disc degeneration frequently manifests as back pain (2). Back pain is the most common cause of disability worldwide. In 2005, the estimated health care cost of this condition in the United States was 85.9 billion dollars (3).

The disc has three components: the annulus fibrosis, the nucleus pulposus and the cartilage endplate. The central gel-like inner nucleus pulposus is primarily composed of the proteoglycan aggrecan (˜65%) and type II collagen (˜15%) (4,5). Cells of the nucleus pulposus are known to be derived from chondrocytes (4). The annulus fibrosis surrounds the nucleus in concentric lamellae. Type I collagen is the main component of the annulus fibrosis (˜70%) (5). In normal discs, proteoglycans and collagen are continually degraded and replaced, creating homeostasis. Degeneration is characterized by both structural and biochemical changes in the disc.

A number of different systems have been designed to characterize the degree of IVD degeneration. A macroscopic grading scale to determine the degree of disc degeneration has been designed (6). Grade I discs are considered normal, with distinct lamellae in the annulus and a uniformly thick end-plate. Grade II discs are categorized by mucinous material between lamellae in the annulus and irregular thickness of the end-plate. The loss of demarcation between the annulus and the nucleus defines a Grade III disc. Grade IV discs are classified by horizontal clefts in the nucleus and focal disruptions in the annulus. Grade V discs are defined by clefts that extend through the nucleus and annulus (6). Several different histologic grading scales measuring the degree of IVD degeneration have also been developed (2,7).

One problem with current therapies is that many very effective anti-inflammatory agents cause cardiac channelopathies or cardiotoxicity. For example, drug induced long QTc Syndrome (LQTS), i.e., a prolongation of the action potential duration is a common cause of governmental mandated drug withdrawal. QTc prolongation is an unpredictable risk factor for Torsades de Pointes (TdP), a polymorphic ventricular tachycardia leading to ventricular fibrillation. Drug induced LQTS comprises about 3% of all prescriptions which when followed by TdP may constitute a lethal adverse reaction. Patients taking one or more than one QTc-prolonging drug concomitantly, have an enhanced risk of TdP. While the overall occurrence of TdP is statistically rare, it is clinically significant for the affected individual. Testing for this drug effect is a mandatory requirement prior to allowing a drug to enter clinical trials.

Common structurally diverse drugs block the human ether-a-go-go-related gene (KCNH2 or hERG) coded K⁺ channel and the cardiac delayed-rectifier potassium current I_{K} (KV11.1) resulting in acquired LQTS. Drug-associated increased risk of LQTS is a major drug development hurdle and many drugs have been withdrawn during pre-clinical development, assigned black box warnings following approval or withdrawn from the market. Autosomal recessive or dominant LQTS based upon 500 possible mutations in 10 different genes coding for the potassium channel has an incidence of 1:3000. Prolonged QT intervals, or risk of LQTS occur in 2.5% of the asymptomatic US population. This syndrome when expressed can lead to severe cardiac arrhythmia and sudden death in untreated patients. The probability of cardiac death in patients with asymptomatic congenital LQTS who are medicated with LQTS-inducing drugs is increased.

The majority of the acquired LTQS drug withdrawals are due to obstruction of the potassium ion channels coded by the human ether-a-go-go related gene (hERG). High concentrations of hERG blocking drugs generally induce a prolonged QTc interval and increase the probability of TdP. Up to 10% of cases of drug-induced TdP can be due to due to 13 major genetic mutations, 471 different mutations, and 124 polymorphisms.

### SUMMARY OF THE INVENTION

In one embodiment, the present invention includes a method of treating an intervertebral disc disease or condition comprising: identifying a patient in need of treatment for the intervertebral disc disease or condition; and administering to the patient an amount of an anti-inflammatory agent that causes a cardiac channelopathy or cardiotoxicity and a liposome, wherein the liposome is provided in an amount sufficient to reduce or eliminate the cardiac channelopathy caused by the anti-inflammatory agent, and the anti-inflammatory agent is provided in an amount sufficient to treat or ameliorate the symptoms of the intervertebral disc disease or condition. In one aspect, the liposomes are defined further as empty liposomes and are provided in an amount sufficient to reduce or eliminate the QT prolongation. In another aspect, the anti-inflammatory agent that causes a cardiac channelopathy is a curcumin or a curcuminoid. In another aspect, the liposome comprises at least one of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, or diacylglycerolsuccinate. In another aspect, the therapeutically effective amount comprises 50 nM/kg, 10 to 100 nM/kg, 25 to 75 nM/kg, 10, 20, 30, 40, 50, 60, 70, 80, 90, or 100 nM/kg curcumin or curcuminoids of body weight of the subject. In another aspect, the curcumin is a synthetic curcumin and is 85, 86, 87, 88, 89, 90, 91, 92, 93, 94, 95 or 96% pure diferuloylmethane. In another aspect, the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione. In another aspect, the curcumin or curcuminoid and the liposomes are adapted to be delivered enterally, parenterally, intravenously, intraperitoneally, or orally. In another aspect, the curcumin or curcuminoid and the liposomes are adapted to be injected intervertebrally. In another aspect, the curcumin or curcuminoid and the liposomes further reduce or eliminate pain caused by the intervertebral disease or condition. In another aspect, the anti-inflammatory agent is selected from at least one of celecoxib; sulindac; oxaprozin; salsalate; diflunisal; piroxicam; indomethacin; etodolac; meloxicam; naproxen; nabumetone; ketorolac tromethamine; naproxen/esomeprazole; serrapeptase; or diclofenac, in an amount that causes a cardiopathy or cardiotoxicity.

In another embodiment, the present invention includes a method of reducing cytokine release and inflammation caused by an intervertebral disc disease or condition comprising: identifying a patient in need of treatment for the intervertebral disc disease or condition; and administering to the patient an amount of a curcumin or a curcuminoid and a liposome sufficient to reduce or ameliorate the cytokine release and inflammation caused by the intervertebral disc disease or condition. In one aspect, the liposomes are defined further as empty liposomes and are provided in an amount sufficient to reduce or eliminate the QT prolongation caused by the curcumin or the curcuminoids. In another aspect, the liposome comprises at least one of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, or diacylglycerolsuccinate. In another aspect, the therapeutically effective amount comprises 50 nM/kg, 10 to 100 nM/kg, 25 to 75 nM/kg, 10, 20, 30, 40, 50, 60, 70, 80, 90, or 100 nM/kg curcumin or curcuminoids of body weight of the subject. In another aspect, the synthetic curcumin is 85, 86, 87, 88, 89, 90, 91, 92, 93, 94, 95 or 96% pure diferuloylmethane. In another aspect, the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione. In another aspect, the curcumin or curcuminoid and the liposomes are adapted to be delivered enterally, parenterally, intravenously, intraperitoneally, or orally. In another aspect, the curcumin or curcuminoid and the liposomes are adapted to be injected intervertebrally. In another aspect, the curcumin or curcuminoid and the liposomes further reduce or eliminate pain caused by the intervertebral disease or condition.

In another embodiment, the present invention includes a method of determining if a candidate drug causes an amelioration symptoms or treats one or more adverse reactions triggered by an intervertebral disc disease or condition in a subject, the method comprising: (a) administering an amount of an anti-inflammatory agent that causes a cardiac channelopathy or cardiotoxicity in combination with empty liposomes, and a placebo to a second subset of the patients, wherein the candidate drug is provided in an amount effective to reduce or prevent the overall level of intervertebral cytokines in the subject; (b) measuring the level of cytokines in the subject from the first and second set of patients; and (c) determining if the anti-inflammatory agent in combination with empty liposomes ameliorates symptoms or treats one or more adverse reactions triggered by the intervertebral disease or condition that triggers a intervertebral cytokines that is statistically significant as compared to any reduction occurring in the subset of patients that took the placebo, wherein a statistically significant reduction indicates that the candidate drug is useful in treating the intervertebral disc disease or condition while also reducing or eliminating the overall level of the intervertebral cytokines. In one aspect, the anti-inflammatory agent is selected from at least one of curcumin; celecoxib; sulindac; oxaprozin; salsalate; diflunisal; piroxicam; indomethacin; etodolac; meloxicam; naproxen; nabumetone; ketorolac tromethamine; naproxen/esomeprazole; serrapeptase; or diclofenac, in an amount that causes a cardiopathy or cardiotoxicity.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In certain embodiments, the compositions have the following abbreviations, chemical names and structures: curcumin (368.38) (MW)

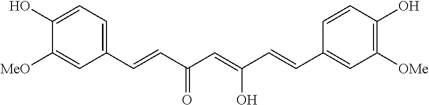 [SMILES: COc1cc(/C=C/C(=O)/C=C(O)/C=C/c2ccc(O)c(OC)c2)ccc1O]

Matrix Metalloproteinases and Pro-Inflammatory Cytokines. Matrix metalloproteinases (MMPs) are a subfamily of zinc-dependent endopeptidases capable of breaking down the extracellular matrix, and which play an important role in disc homeostasis (8) MMPs and aggrecanases are involved in the degradation of proteoglycans and collagen. The collagenases (MMP-1, MMP-8, MMP13) cleave the fibrillar collagens, types I, II and III. Proteoglycans are degraded by the gelatinases (MMP-2, MMP-9) and stromelysin (MMP-3) (9, 10). Tissue inhibitors of metalloproteinases (TIMPs) inactivate MMPs. Therefore, the balance of MMPs to TIMPs is crucial for normal disc homeostasis (11).

Cardiac Channelopathies and cardiotoxicity. The human ether-à-go-go gene related cardiac tetrameric potassium channel, which when mutated can render patients sensitive to over 163 drugs which may inhibit ion conduction and deregulate action potentials. Prolongation of the action potential follows effects in the potassium channel. Ion channel active drugs may directly increase the QTc interval, and increase the risk of torsade de point and sudden cardiac death. Exacerbation of cardiomyocyte potassium channel sensitivity to drugs may also be associated with metabolic diseased states including diabetes or may be of idiopathic origin.

The mechanism of human ether-à-go-go related gene channels blockade may be analogous to the effects of externally applied quaternary ammonium derivatives which indirectly may suggest the mechanism of action of the anti-blockading effect of the DMPC/DMPG liposome or its metabolites. The inhibitory constants and the relative binding energies for channel inhibition indicate that more hydrophobic quaternary ammoniums have higher affinity blockade while cation-π interactions or size effects are not a deterministic factor in channel inhibition by quaternary ammoniums. Also hydrophobic quaternary ammoniums either with a longer tail group or with a bigger head group than tetraethylammonium permeate the cell membrane to easily access the high-affinity internal binding site in the gene channel and exert a stronger blockade.

In intervertebral disc (IVD) disease, homeostasis is disturbed and an increase in MMP activity is observed (8). Although studies of MMPs in IVD disease have been done utilizing a variety of experimental methods, they have consistently shown evidence of increased MMP activity in models of this disease. Increased MMP activity leads to a loss of proteoglycans and consequently to changes in the disc (12). Crean et al associated the levels of MMP-2 and MMP-9 with the macroscopic degree of disc degeneration (9). Grade IV samples showed four times greater activity of MMP-2 than did grade II samples. Likewise, MMP-9 activity was three times greater in grade IV samples compared to grade II samples (Thompson grading scale). In samples from patients with degenerated discs, herniated discs or spondylolisthesis, Roberts et al reported that extensive staining for MMP-1 could be detected in 91% of samples, for MMP-2 in 71%, for MMP-3 in 65%, for MMP-7 in 35%, for MMP-8 in 35%, and for MMP-9 in 72% (10). In another study, Nemoto et al showed that cultured IVD cells from patients with degenerated discs produced two times more MMP-3 than did cells from patients with normal discs (13). Kang et al found four times higher MMP-3 activity levels in herniated discs compared to control discs (acute trauma patients) (14). Wei et al found almost nine times greater expression of MMP-3 in the intervertebral discs of rhesus monkeys after degeneration had been induced by bleomycin vs. controls (15). Studies showing abnormal MMP activity in IVD disease are listed in Table 1.

TABLE 1
Activity of Key MMPs and TIMPs in Degenerated Discs

| Biomolecule | Model | Comment |
|---|---|---|
| MMP-1 | Quantitative RT-PCR. Degenerated | ~38% showed at least 50 fold increase in |
| | IVD vs. controls (acute trauma) | expression* |
| | IHC. Degenerated IVD vs. controls | Mean 55% positive cells |
| | (acute trauma) | |
| | IHC. Prolapsed IVD vs. controls | Occurs in 100% of prolapsed samples vs. |
| | (autopsy) | 86% of controls |
| | IHC. IVD with varying grades of | Mean expression in AF 1.5x greater in |
| | degeneration | highly degenerated discs vs. moderately |
| | | degenerated discs* |
| | IHC. IVD with varying grades of | ~80% immunoreactive cells from NP of |
| | degeneration | discs intermediate degenerative score; |
| | | ~50% immunoreactive cells from NP of |
| | | discs with low-degenerative score* |
| MMP-2 | Quantitative RT-PCR. Degenerated | ~46% showed at least 50 fold increase in |
| | IVD vs. controls (acute trauma) | expression* |
| | Quantitative zymography. | 4x greater activity in grade IV samples |
| | Degenerated IVDs | vs grade II samples (Thompson scale)* |
| | IHC. Prolapsed IVD vs. controls | Occurs in 100% of prolapsed samples vs. |
| | (autopsy) | 57% of controls |
| | IHC. IVD with varying grades of | Mean expression in AF ~5x greater in |
| | degeneration | highly degenerated discs vs. moderately |
| | | degenerated discs* |
| MMP-3 | Enzyme immunoassay. Degenerated | 2x increase over controls (p < 0.05) |
| | vs. normal IVD. | |
| | IHC. Prolapsed IVD vs. controls | Occurs in 89% of prolapsed samples vs. |
| | (autopsy) | 57% of controls |
| | Quantitative RT-PCR. Degenerated | ~27% showed >5000 fold increase in |
| | IVD vs. controls (acute trauma) | expression; |
| | | ~48% showed 500-5000 fold increase in |
| | | expression; |
| | | (~93% showed at least 50 fold increase)* |
| | IHC. Degenerated IVD vs. controls | Mean 65% positive cells |
| | (acute trauma patients) | |
| | Enzyme immunoassay. Herniated | 4x greater production of MMP-3 vs. |
| | IVD vs. controls (surgery for | controls (p < 0.0024) |
| | scoliosis or trauma) | |
| | Real time PCR. IVD rhesus | ~9x greater expression vs. controls* |
| | monkeys injected with bleomycin | |
| | vs. controls (normal IVD rhesus | |
| | monkeys) | |
| | Immunocapture activity assay. | High concentrations of MMP-3. MMP-3 |
| | Herniated disc tissue | levels >30x greater than MMP-1(p < 0.01) |
| | IHC. IVD with varying grades of | ~5% immunoreactive cells from NP of |
| | degeneration | discs with low-degenerative score; ~10% |
| | | immunoreactive cells from NP of discs |
| | | with high degenerative score* |
| MMP-7 | IHC. Prolapsed IVD vs. controls | Occurs in 63% of prolapsed samples vs. |
| | (autopsy) | 29% of controls |
| | IHC. IVD with varying grades of | Non-degenerate IVD: ~6% |
| | degeneration | immunopositive cells; |
| | | Intermediate degeneration: ~18% |
| | | immunopositive cells; |
| | | Severe degeneration: ~50% |
| | | immunopositive cells; |
| | | Prolapsed IVD: ~22% immunopositive |
| | | cells |
| MMP-8 | Quantitative RT-PCR. Degenerated | ~43% showed at least 500 fold increase |
| | IVD vs. controls (acute trauma) | in expression; |
| | | ~92% showed at least 50 fold increase* |
| | IHC. Prolapsed IVD vs. controls | Occurs in 56% of prolapsed samples vs. |
| | (autopsy) | 14% of controls |
| MMP-9 | Quantitative zymography. | 3x greater activity in grade IV samples |
| | Degenerated IVD | vs. grade II samples (Thompson scale)* |
| | IHC. Prolapsed IVD vs. controls | Occurs in 79% of prolapsed samples vs. |
| | (autopsy) | 43% of controls |
| | Quantitative RT-PCR. Degenerated | ~72% showed 50 fold increase* |
| | IVD vs. controls (acute trauma) | |
| MMP-10 | Quantitative RT-PCR. Surgical | 15x increase in surgical degenerated |
| | degenerated discs vs. controls | discs vs. controls* |
| | (postmortem normal discs) | |
| MMP-13 | Quantitative RT-PCR. Degenerated | ~45% showed 50 fold increase* |
| | IVD vs. controls (acute trauma) | |
| MMP-28 | IHC. High grade discs vs. low grade | Found in extracellular matrix of 61% of |
| | discs (Thompson scale) | grade III-V discs vs. 0% of grade I and II |
| | | discs |
| TIMP-1 | Quantitative RT-PCR. Degenerated | ~65% showed at least 5000 fold increase |
| | IVD vs. controls (acute trauma) | in expression |
| | | ~95% showed at least 50 fold increase in |
| | | expression* |
| | IHC. Prolapsed IVD vs. controls | Expressed in 61% of prolapsed samples |
| | (autopsy) | vs. 0% of controls |
| | IHC. IVD with varying grades of | ~5% immunoreactive cells from NP of |
| | degeneration | non-degenerated discs; 40% |
| | | immunoreactive cells from NP of discs |
| | | high degenerative score* |
| TIMP-2 | Quantitative RT-PCR. Degenerated | ~65% showed at least 500 fold increase |
| | IVD vs. controls (acute trauma) | in expression* |
| | IHC. Prolapsed IVD vs. controls | Expressed in 78% of prolapsed samples |
| | (autopsy) | vs. 86% of controls |
| | IHC. IVD with varying grades of | ~40% immunoreactive cells from NP of |
| | degeneration | discs with high degenerative score; ~5% |
| | | immunoreactive cells from NP of non- |
| | | degenerated discs* |

RT-PCR: Real time quantitative reverse transcription polymerase;
IHC: Immunohistochemistry;
NP: Nucleus pulposus;
AF: Annulus fibrosis
*Exact values not given

Pro-inflammatory cytokines are known to up-regulate MMPs. Interleukin 1β (IL-1β), interleukin 6 (IL-6), interleukin 8 (IL-8) and tumor necrosis factor α (TNF-α) increase MMP activity. Numerous studies have shown increased levels of these cytokines compared to controls in degenerated discs, and, further, higher levels in discs that were more degenerated. Lemaitre et al found greater levels of both TNF-α and IL-1β in degenerated or herniated discs than in non-degenerated discs. TNF-α was expressed by 96% of the degenerated IVDs, whereas only 13% of non-degenerated IVDs expressed this cytokine (16). 100% of degenerated discs expressed IL-1β compared to 63% of non-degenerated discs. Burke et al showed significantly higher levels of IL-6, IL-8 and prostaglandin E₂ (PGE₂) in IVD discs that showed greater degeneration. 90% of the discs from low back pain patients where nuclear extrusion was present produced IL-6, IL-8 and PGE₂ compared to 50% of discs from low back pain patients where the annulus was intact. A linear relationship between IL-6 production and IL-8 production was also noted (17). Shamji et al showed that both herniated IVDs and degenerated IVDs showed greater expression of IL-6, IL-12 and Il-17 than did autopsy controls (18). Weiler et al showed degenerated discs had greater expression levels of TNF-α than the controls (19). Most importantly, discs with a higher degree of degeneration had higher expression level of TNF-α (Boos scale). TNF-α was expressed in 60% of cells from discs with a histological degeneration score (HDS) of 4, in 40% of cells from discs in the HDS group 3, 19% in cells from discs in the HDS group 2 and in <5% in controls (19). (See Table 2).

TABLE 2
Activity of Key Cytokines in Degenerated Discs

| Biomolecule | Model | Comment |
|---|---|---|
| IL-1β | IHC. Human degenerated IVD vs. | Expressed in 100% of degenerated IVD vs. |
| | controls (autopsy) | 63% of controls |
| | Enzyme immunoassay. Low back | Not detected |
| | pain patients,: IVD with nuclear | |
| | extrusion vs IVD with intact | |
| | annulus | |
| | Enzyme immunoassay. | Not detected |
| | Degenerated IVD vs. controls | |
| | (surgery for scoliosis or trauma) | |
| | Real time PCR. IVD rhesus | ~6x greater expression vs. controls* |
| | monkeys injected with bleomycin | |
| | vs. controls (normal IVD rhesus | |
| | monkeys) | |
| | Real Time- PCR. Degenerated | ~4x greater relative mRNA expression vs. |
| | IVD vs. controls (acute fracture) | controls* |
| | Immunofluorescent Painting. | 31.61 ± 7.82 pg/ml vs. controls 11.45 ± 3.80 |
| | Degenerated IVD vs. controls | pg/ml (p = 0.0001) |
| | (autopsy) | |
| IL-2 | Immunofluorescent Painting. | 11.88 ± 2.51 pg/ml vs. controls 3.92 ± 1.13 |
| | Degenerated IVD vs. controls | pg/ml (p = 0.0001) |
| | (autopsy) | |
| IL-4 | IHC. Degenerated IVD vs. | Herniated IVD: immunoreactivity in ~20% |
| | Herniated IVD vs. controls | of fields |
| | (autopsy) | Degenerated IVD: immunoreactivity in |
| | | ~10% of fields |
| | | Controls: immunoreactivity in <5% of |
| | | fields* |
| | Immunofluorescent Painting. | 32.18 ± 10.38 pg/ml vs. controls 8.37 ± 3.35 |
| | Degenerated IVD vs. controls | pg/ml (p = 0.0001) |
| | (autopsy) | |
| IL-6 | Enzyme immunoassay. Low back | ~1.5-2x greater production in IVD with |
| | pain patients: IVD with nuclear | nuclear extrusion* |
| | extrusion vs IVD with intact | |
| | annulus | |
| | IHC. Degenerated IVD vs. | Herniated IVD: immunoreactivity in 30% |
| | Herniated IVD vs. controls | of fields; |
| | (autopsy) | Degenerated IVD: immunoreactivity in |
| | | 10% of fields; |
| | | Controls: immunoreactivity in <5% of |
| | | fields* |
| | Enzyme immunoassay. Herniated | Mean 30,000 Pg/ml vs. barely detectable |
| | IVD vs. controls (surgery for | levels in controls |
| | scoliosis or trauma) | |
| | Real time PCR. IVD rhesus | ~7x greater expression vs. controls* |
| | monkeys injected with bleomycin | |
| | vs. controls (normal IVD rhesus | |
| | monkeys) | |
| | Real Time- PCR. Degenerated | No significant change vs. controls |
| | IVD vs. controls (acute fracture) | |
| IL-8 | Enzyme immunoassay. Low back | ~1.5-2x greater production in IVD with |
| | pain patients: IVD with nuclear | nuclear extrusion* |
| | extrusion vs IVD with intact | |
| | annulus | |
| | RT-PCR. Herniated IVD | Expressed in 70% of specimens. |
| | | Associated with development of radicular |
| | | pain |
| IL-12 | IHC. Degenerated IVD vs. | Herniated IVD: immunoreactivity in 20% |
| | Herniated IVD vs. controls | of fields; |
| | (autopsy) | Degenerated IVD: immunoreactivity in |
| | | ~10% of fields*; |
| | | Controls: immunoreactivity in <5% of |
| | | fields (NP, AF) |
| | Immunofluorescent Painting. | 7.33 ± 2.15 pg/ml vs. controls 4.09 ± 1.04 |
| | Degenerated IVD vs. controls | pg/ml (p = 0.0001) |
| | (autopsy) | |
| IL-16 | Real Time- PCR. Degenerated | ~5x greater relative mRNA expression vs. |
| | IVD vs. controls (acute fracture) | controls* |
| IL-17 | IHC. Degenerated IVD vs. | Herniated IVD: immunoreactivity in 90% |
| | Herniated IVD vs. controls | of fields; |
| | (autopsy) | Degenerated IVD: immunoreactivity in |
| | | ~70% of fields*; |
| | | Controls: immunoreactivity in 50% of |
| | | fields (NP), 0% (AF) |
| | Immunolocalization. Degenerated | Greater expression in more degenerated |
| | IVD vs. controls | discs. No difference between herniated and |
| | | and non-herniated discs |
| TNF-α | IHC. Degenerated IVD vs. | Expressed in 96% of degenerated IVD vs. |
| | controls (autopsy) | 13% of controls |
| | Enzyme immunoassay. IVD from | Not detected |
| | patients undergoing surgery for | |
| | sciatica or back pain | |
| | Enzyme immunoassay. | Not detected |
| | Degenerated IVD vs. controls | |
| | (surgery for scoliosis or trauma) | |
| | IHC. Degenerated IVD vs. | 60% expression HDS 4, 40% HDS 3, 19% |
| | controls (autopsy) | HDS 2, <5% controls |
| | Real time PCR. IVD rhesus | ~5x greater expression vs. controls* |
| | monkeys injected with bleomycin | |
| | vs. controls (normal IVD rhesus | |
| | monkeys) | |
| | Real Time- PCR. Degenerated | ~1.5x greater relative mRNA expression |
| | IVD vs. controls (acute fracture) | vs. controls* |
| Interferon-γ | IHC. Degenerated IVD vs. | Herniated IVD: immunoreactivity in ~40% |
| | Herniated IVD vs. controls | of fields*; |
| | (autopsy) | Degenerated IVD: immunoreactivity in |
| | | ~10% of fields*; |
| | | Controls: immunoreactivity in <5% of |
| | | fields (NP, AF) |
| | Immunofluorescent Painting. | 8.16 ± 3.95 pg/ml vs. controls 5.61 ± 1.83 |
| | Degenerated IVD vs. controls | pg/ml (p = 0.054) |
| | (autopsy) | |

IHC: Immunohistochemistry;
PCR: Polymerase chain reaction;
HDS: Histological degeneration score;
NP: Nucleus pulposus;
AF: Annulus fibrosis
*Exact values not given

Curcumin's Suppresses MMPs in Multiple Disease States.

The phytochemical curcumin, the principle component of turmeric, has numerous anti-inflammatory, anti-viral and anti-cancer effects (20, 21). Curcumin is one example of a composition that is known to cause cardiopathy if injected intravenously, e.g., QT prolongation. Curcumin is known to inhibit the expression of MMPs. Hassan et al showed that curcumin inhibited both MMP-2 and MMP-9 in a MDA breast cancer cell line. The same study found TIMP-1, TIMP-2, TIMP-3 and TIMP-4, suppressors of MMPs, were up-regulated by high concentrations of curcumin (22). Similarly, Shao et al showed curcumin down-regulated MMP-2 and up-regulated TIMP-1 in estrogen receptor-negative MDA-MB-231 breast cancer cells (23). Inhibition of MMP-2 and MMP-9 by curcumin was also observed by Lin et al in a human non-small cell lung cancer cell line (A549) in vitro (24). Epstein et al studied colonic myofibroblasts from patients with inflammatory bowel disease (25). When curcumin was added to the cell culture, a dose dependent suppression of MMP-3 was seen. Kundu et al investigated human gastric epithelial cells infected with helicobacter pylori, and found that curcumin suppressed MMP-3 and MMP-9 activity (26). This effect was dose dependent. Banerji et al reported on curcumin's effects on B16F10 metastatic melanoma cells in a mouse model (27). Suppression of MMP-1, MMP-3 and MMP-9 by curcumin was also reported in human astroglioma cells (28). Mun et al administered curcumin orally to mice with collagen-induced arthritis. Curcumin was shown to suppress type II collagen-induced arthritis in these mice, and MMP-1 and MMP-3 expression in their joints, in a dose dependent manner (29).

Curcumin Suppression of Pro-Inflammatory Cytokines. Curcumin has been shown to inhibit the release of pro-inflammatory cytokines in multiple experimental models. Curcumin suppresses IL-1β, IL-8, TNF-α, monocyte chemoattractant protein-1 (MCP-1) and macrophage inflammatory protein-1α (MIP-1α) release from monocytes and macrophages (30). The release of IL-6, IL-8, TNF-α and MCP-1 from monocytes that had been cultured in a high glucose environment was markedly reduced by curcumin (31). Yu et al showed curcumin's suppression of TNF-α levels in an acute pancreatitis mouse model was associated with decreased pancreatic injury (32). Gulcubuk et al reported that curcumin reduced TNF-α and IL-6 levels in Wistar albino rats with experimental acute pancreatitis (33). Curcumin's effect on cytokine expression and disease progression in a mouse model of viral induced acute respiratory distress syndrome was reported by Avasarala et al. Curcumin reduced the expression of key cytokines IL-6, IL-8, interferon-γ and MCP-1, and this correlated with a marked decrease in inflammation and reduction in fibrosis (34). Curcumin has also been reported to block the release of IL-6 in rheumatoid synovial fibroblasts, as well as the release of IL-8 in human esophageal epithelial cells and human articular chondrocytes (35, 36). Zhang et al showed that curcumin reduced IL-6, IL-8 and TNF-α expression in rats with non-bacterial prostatitis (37). Gao et al found production of interferon-γ and expression of IL-2 in splenic T lymphocytes were inhibited by curcumin. The same study also showed curcumin inhibited production of TNF-α and expression of IL-12 in peritoneal macrophages (38). Curcumin also suppressed pro-inflammatory cytokines in pancreatic carcinoma (39), hepatocellular carcinoma (40), colon carcinoma (41) and multiple myeloma (42, 43) cell lines.

Curcumin's Effect on Chondrocytes and IVD Cells. Several compounds derived from natural products have been investigated for the treatment of intervertebral disc degeneration. Krupkova et al studied the effects of epigallocatechin 3-gallate (EGCG), a polyphenol of green tea, on human nucleus pulposus tissue from patients with IVD degeneration. In this study, the IVD cells were stimulated with IL-1β, and then treated with 10 μM EGCG. It was found that EGCG inhibited the expression of IL-6, IL-8, COX-2, MMP-1, MMP-3 and MMP-13 in these cells (44). Similar results were found by Wuertz et al using resveratrol, a polyphenol of red wine. Resveratrol reduced the levels of IL-6, IL-8, MMP-1, MMP-3 and MMP-13 in IVD cells pre-treated with IL-1β (45).

Genevay et al studied the effects of cytokine inhibitors and glucocorticoids on MMP-1 and MMP-3 activity in the IVD of patients undergoing lumbar discectomy for back pain. Samples were treated with recombinant interleukin-1 receptor antagonist (IL-1Ra), TNF inhibitor monoclonal antibody, or dexamethasone. MMP-1 and MMP-3 activity were determined by immunocapture activity assays. The authors reported that dexamethasone markedly decreased concentrations of both MMP-1 and MMP-3; the concentration of total MMP-1 activity was decreased from 0.03 ng/mg tissue to 0.004 ng/mg tissue and the concentration of total MMP-3 activity decreased from 0.9 ng/mg tissue to 0.46 ng/mg tissue. The concentration of total MMP-3 activity decreased to 0.35 ng/mg tissue in the IL-1Ra treated samples, and decreased to 0.3 ng/mg tissue in the TNF inhibitor treated samples. However, no effect on MMP-1 concentration was seen in samples treated with IL-1Ra or TNF inhibitor (46).

Curcumin has multiple effects on articular chondrocytes. Shakibaei et al treated chondrocytes with curcumin, and noted a decrease in IL-1 induced NF-κB activation and a down regulation of COX2 and MMP-9 (47). Clutterbuck et al studied curcumin's effects on equine articular cartilage explants and on equine chondrocytes. In the cartilage explants, curcumin reduced IL-1β stimulated MMP-3 release (48). This effect was dose dependent. Similar results were found by Schulze-Tanzil et al, who showed curcumin inhibited MMP-3 activity in IL-1β stimulated chondrocytes (49). Csaki et al found that human articular chondrocytes co-treated with curcumin and resveratrol inhibited IL-1β induced NF-κB activation (50). Curcumin was also shown to suppress TNF-α induced MMP-13 expression in primary chondrocytes (51).

The effect of curcumin on pro-inflammatory cytokines and MMPs in cultured degenerated human intervertebral disc cells has also been reported. Yu et al studied the IVD cells of patients with acute spinal injury, but no history of lower back pain. Two groups of cells were stimulated with IL-1, and one was subsequently treated with curcumin. It was found that the IL-1-only group expressed NF-κB in a dose dependent manner (52). However, IL-1 induced NF-κB activity was blocked in the cells treated with curcumin. Yu et al also found that curcumin reversed the IL-1 inhibition of SOX9 and collagen type II expression (52).

In a similar study, Klawitter et al examined the effects of curcumin DMSO extract and curcumin ethanol extract on human intervertebral disc cells. The disc cells were pre-stimulated with IL-1β, and then treated with curcumin extract (53). The cells treated with curcuma DMSO showed marked reductions in MMP-1, MMP-3 and MMP-13 expression. A 22% reduction in MMP-1 expression was seen after 5 μM treatment of curcumin, a 60% reduction after 10 μM of curcumin and a 78% reduction with 20 μM of curcumin. MMP-3 expression was reduced 70% with 10 μM of curcumin, and reduced 75% with 20 μM. MMP-13 expression decreased 55% with 5 μM curcumin; a 90% reduction was seen with 10 μM and with 20 μM curcumin. Expression of IL-1β was reduced 70% with 10 μM of curcumin and 75% with 20 μM of curcumin. Curcumin also decreased the expression of IL-6 by 50% at 10 μM and by 70% at 20 μM. Similarly, IL-8 expression was reduced 50% with curcumin at 20 μM. Studies showing the suppressive effects of curcumin on MMPs and pro-inflammatory cytokines are given in Tables 3 and 4.

TABLE 3
Curcumin Suppression of Matrix Metalloproteinases (MMPs)

| Molecule | Function |
|---|---|
| MMP-1 (Collagenase-1) | Collagenase; degrades fibrillar collagen types I, |
| | II and III |
| MMP-2 (Gelatinase A) | Gelatinase |
| MMP-3 (Stromelysin-1) | Cleaves extracellular matrix proteins, proteo- |
| | glycans, fribronectin elastin |
| MMP-7 (Matrilysin) | Breaks down extracellular matrix, proteogly- |
| | cans |
| MMP-8 (Neutrophil | Degrades type I, II, III collagens |
| Collagenase) | |
| MMP-9 (Gelatinase-B) | Collagenase; gelatinase |
| MMP-10 (Stromelysin-2) | Degrades proteoglycans and fibronectin |
| MMP-12 | Breaks down extracellular matrix, elastin |
| MMP-13 (Collagenase-3) | Degrades type IV, V collagens |
| MMP-14 | Cleaves fibrillar collagen; breaks down |
| | extracellular matrix |

*Curcumin effect on IVD
** Curcumin effect on chondrocytes

TABLE 4
Curcumin has been shown to suppress pro-inflammatory
cytokines in numerous experimental models

| Cytokine | Function |
|---|---|
| TNF-α | Major pro-inflammatory cytokine; |
| | insulin resistance; induces secretion of |
| | corticotropin-releasing factor; |
| | upregulates MMP expression; |
| | stimulates chondrocytes to produce |
| | chemokines; reduces glycoprotein and |
| | collagen synthesis |
| IL-1β | Major pro-inflammatory cytokine; |
| | hematopoiesis; CNS development; |
| | upregulates MMP expression; induces |
| | chondrocyte apoptosis; inhibits |
| | proteoglycan synthesis |
| IL-2 | T-cell lymphocyte differentiation |
| IL-4 | B-cell lymphocyte proliferation |
| IL-6 (Interferon β2) | Major pro-inflammatory cytokine; B- |
| | cell lymphocyte differentiation; nerve |
| | cell differentiation; increases MMP-2 |
| | activity; inhibits chondrocyte |
| | proliferation; stimulates aggrecanase- |
| | mediated proteoglycan catabolism |
| IL-8 (CXCL8) | Neutrophil chemotaxis; angiogenesis; |
| | chondrocyte calcification |
| IL-12 | Defense against intracellular pathogens |
| IL-17 | Pro-inflammatory cytokine; induces |
| | MMP production |
| Interferon-γ | Macrophage activation; T and B cell |
| | activation and differentiation |

*Curcumin effect on IVD,
** Curcumin effect on chondrocytes

Only one study of curcumin in patients with IVD disease has been done. Di Pierro et al treated patients with lumbar herniation with a dexibuprofen and a tablet containing lipoic acid plus curcumin and piperine. The authors observed a 70% reduction in patient-related symptoms of peripheral neuropathy. However, the levels of MMPs and cytokines were not measured in these patients (54).

Increased MMP activity and an increase in pro-inflammatory cytokines are associated with an increased degree of intervertebral disc degeneration. Curcumin, a known anti-inflammatory agent, has been studied in a number of experimental models and has been shown to have suppressant effects on both MMPs and cytokines. Higher concentrations of curcumin seem to have more profound effects. As such, curcumin with reduced or no toxicity can be used in patients with IVD disease. The present invention takes advantage of the inventors' discovery that liposomes can reduce or eliminate cardiac channelopathies caused by inflammatory agents that have, as a negative side-effect, cardiac channelopathies, such as curcumin. The present invention includes the use of an anti-inflammatory agent that causes a cardiac channelopathy and a liposome, wherein the liposome is provided in an amount sufficient to reduce or eliminate the cardiac channelopathy caused by the anti-inflammatory agent, and the anti-inflammatory agent is provided in an amount sufficient to treat or ameliorate the symptoms of the intervertebral disease or condition.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Miller J A A, Schmatz C, Schultz A B. Lumbar disc degeneration: Correlation with age, sex, and spine level in 600 autopsy specimens. Spine. 1988; 13:173-178.
2. Boos N, Weissbach S, Rohrbach H, Weiler C, Spratt K F, Nerlich A G. Classification of age-related changes in lumbar intervertebral discs. Spine. 2002; 27:2631-2644.
3. Martin B I, Deyo R A, Mirza S K, Turner J A, Comstock B A, Hollingworth W and Sullivan S D: Expenditures and health status among adults with back and neck problems. JAMA. 2008; 299:656-664.
4. Freemont A J. The cellular pathobiology of the degenerate intervertebral disc and discogenic back pain. Rheumatology. 2009; 48:5-10.
5. Urban J P G, Roberts S. Degeneration of the intervertebral disc. Arthritis Research and Therapy. 2003; 5:120-130.
6. Thompson J P, Pearce R H, Schechter M T, Adams M E, Tsang I K Y and Bishop P B. Preliminary evaluation of a scheme for grading the gross morphology of the human intervertebral disc. Spine. 1990; 15:411-415.
7. Gries N C, Berlemann U, Moore R J and Vernon-Roberts B. Early histologic changes in lower lumbar discs and facet joints and their correlation. Eur Spine J. 2000; 9:23-29.
8. Roberts S, Caterson B, Menage J, Evans E H, Jaffray D C, Eisenstein S M. Matrix metalloproteinases and aggrecanase: Their role in disorders of the human intervertebral disc. Spine. 2000; 25:3005-3013.
9. Crean J K G, Roberts S, Jaffray D C, Eisentein S M, Duance V C. Matrix metalloproteinases in the human intervertebral disc: Role in disc degeneration and scoliosis. Spine. 1997; 22:2877-2884.
10. Roberts S, Evans H, Trivedi J, Menage J. Histology and pathology of the human intervertebral disc. The Journal of Bone and Joint Surgery. 2006; 88-A: Supplement 2.
11. Bachmeier B E, Nerlich A, Mittermaier N, Weiler C, Lumenta C, Wuertz Boos N. Matrix metalloproteinase expression levels suggest distinct enzyme roles during lumbar disc herniation and degeneration. Eur Spine J. 2009; 18:1573-1586.
12. Wuertz K, Vo N, Kletas D, Boos N. Inflammatory and catabolic signaling in intervertebral discs: The roles of NF-κB and MAP kinases. European Cells and Materials. 2012; 23:103-120.
13. Nemoto O, Yamagishi M, Yamada H, Kikuchi T, Takaishi H. Matrix metalloproteinase-3 production by human degenerated intervertebral disc. Journal of spinal disorders. 1997; 10:493-498.
14. Kang J D, Georgescu H I, McIntyre-Larkin L, Stefanovic-Racic M, Donaldson W F and Evans C H: Herniated lumbar intervertebral discs spontaneously produce matrix metalloproteinases, nitric oxide, interleukin-6, and prostaglandin E2. Spine. 1996; 21:271-277.
15. Wei F, Zhong R, Zhou Z, Wang L, Pan X, Cui S, Zou X, Gao M, Sun H, Chen W, Liu S. In vivo experimental intervertebral disc degeneration induced by bleomycin in the rhesus monkey. BMC Muskuloskeletal Disorders. 2014; 15:340.
16. Le Maitre C L, Hoyland J A, Freemont A J. Catabolic cytokine expression in degenerate and herniated human intervertebral discs: IL-1β and TNF-α expression profile. Arthritis Research and Therapy. 2007; 9:R77.
17. Burke J G, Watson R W G, McCormack D, Dowling F E, Walsh M G, Fitzpatrick J M. Intervertebral discs which cause low back pain secrete high levels of proinflammatory mediators. J Bone Joint Surg. 2002; 84-B: 196-201.
18. Shamji M F, Setton L A, Jarvis W, So S, Chen J, Jing L, Bullock R, Isaacs R E, Brown C, Richardson W J. Proinflammatory cytokine expression profile in degenerated and herniated human intervertebral disc tissues. Arthritis and Rheumatism. 2010; 62:1974-1982.
19. Weiler C, Nerlich A G, Bachmeier B E and Boos N: Expression and distribution of tumor necrosis factor alpha in human lumbar intervertebral discs: A study in surgical specimen and autopsy controls. Spine. 2004; 30:44-54.
20. Basnet P, Skalko-Basnet N. Curcumin: An anti-inflammatory molecule from a curry spice on the path to cancer treatment. Molecules. 2011; 16:4567-4598.
21. Sordillo P P, Helson L. Curcumin suppression of cytokine release and cytokine storm. A potential therapy for patients with Ebola and other severe viral infections. In Vivo. 2015; 29: 1-4.
22. Hassan Z K, Daghestani M H. Curcumin effect on MMPs and TIMPs genes in a breast cancer cell line. Asian Pac J Cancer Prev. 2012; 13:3259-3264.
23. Shao Z M, Shen Z Z, Liu C H, Sartippour M R, Go V L, Herber D, Nguyen M. Curcumin exerts multiple suppressive effects on breast carcinoma cells. International Journal of Cancer. 2001; 98:234-240.
24. Lin S S, Lai K C, Hsu S C, Yang J S, Kuo C L, Lin J P, Ma Y S, Wu C C and Chung J G, Curcumin inhibits the migration and invasion of human A549 lung cancer cells through the inhibition of matrix metalloproteinase-2 and -9 and vascular endothelial growth factor (VEGF). Cancer Letters. 2009; 285:127-133.
25. Epstein J, Docena G, MacDonald T T and Sanderson I R: Curcumin suppresses p38 mitogen-activated protein kinase activation, reduces IL-1β and matrix metalloproteinase-3 and enhances IL-10 in the mucosa of children and adults with inflammatory bowel disease. British Journal of Nutrition. 2010; 103:824-832.
26. Kundu P, De R, Pal I, Mukhopadhyay A K, Saha D R, Swarnakar S. Curcumin alleviates matrix metalloproteinase-3 and -9 activities during eradication of Helicobacter pylori infection in cultured cells and mice. PLoS ONE. 2011; 6 (1):e16306. doi:10.1371/journal.pone.0016306.
27. Banerji A, Chakrabarti J, Mitra A, Chatterjee A. Effect of curcumin on gelatinase A (MMP-2) activity in B16F10 melanoma cells. Cancer Letters. 2004; 211:235-242.
28. Kim S Y, Jung S H, Kim H S. Curcumin is a potent broad spectrum inhibitor of matrix metalloproteinase gene expression in human astroglioma cells. Biochemical and Biophysical Research Communications. 2005; 337:510-516.
29. Mun S H, Kim H S, Kim J W, Ko N Y, Kim D K, Lee B Y, Kim B, Won H S, Shin H S, Han J W, Le H Y, Kim Y M, Choi W S. Oral administration of curcumin suppresses production of matrix metalloproteinase (MMP)-1 and MMP-3 to ameliorate collagen-induced arthritis: Inhibition of the PKCδ/JNK/c-Jun pathway. J Pharmacol Sci. 2009; 111:13-21.
30. Abe Y, Hashimoto S and Horie T: Curcumin inhibition of inflammatory cytokine production by human peripheral blood monocytes and alveolar macrophages. Pharmacol Res. 1999; 39:41-47.
31. Jain S K, Rains J, Croad J, Larson B and Jones K: Curcumin supplementation lowers TNF-α, IL-6, IL-8, and MCP-1 secretion in high glucose-treated cultured monocytes and blood levels of TNF-α, IL-6, MCP-1, glucose, and glycosylated hemoglobin in diabetic rats. Antioxid Redox Signal. 2009; 11: 241-249.
32. Yu W G, Xu G, Ren G J, Xu X, Yuan H Q, Qi X L and Tian K L: Preventive action of curcumin in experimental acute pancreatitis in mouse. Indian J Med Res. 2011; 134:717-724.
33. Gulcubuk A., Altunatmaz K., Sonmez K., Haktanir-Yatkin D., Uzun H., Gurel A., Aydin S. Effects of curcumin on tumour necrosis factor-alpha and interleukin-6 in the late phase of experimental acute pancreatitis. J Vet Med A Physiol Pathol Clin Med. 2006; 53:49-54.
34. Avasarala S, Zhang F, Liu G, Wang R, London S D and London L: Curcumin modulates the inflammatory response and inhibits subsequent fibrosis in a mouse model of viral-induced acute respiratory distress syndrome. PLoS ONE http://dx.doi.org/10.1371/journal.pone.2013.0057285.
35. Kloesch B, Becker T, Dietersdorfer E, Kiener H and Steiner G: Anti-inflammatory and apoptotic effects of the polyphenol curcumin on human fibroblast-like synoviocytes. Int Immunopharmacol. 2013; 15:400-405.
36. Raflee P, Nelson V M, Manley S, Wellner M, Floer M, Binion D G and Shaker R: Effect of curcumin on acidic pH-induced expression of IL-6 and IL-8 in human esophageal epithelial cells (HET-1A): Role of PKC, MAPKs, and NF-κB. Amer J Physiol-Gastrointest Liver Physiol. 2009; 296:G388-G398.
37. Zhang Q Y., Mo Z N., Liu X D. Reducing effect of curcumin on expressions of TNF-alpha, IL-6 and IL-8 in rats with chronic nonbacterial prostatitis. National Journal of Andrology. 2010; 16:84-88.
38. Gao X, Kuo J, Jiang H, Deeb D, Liu Y, Divine G, Chapman R A, Dulchaysky S A and Gautam S C: Immunomodulatory activity of curcumin: Suppression of lymphocyte proliferation, development of cell-mediated cytotoxity, and cytokine production in vitro. Biochem Pharmacol. 2004; 68:51-61.
39. Bisht S., Feldmann G., Soni S., Ravi R., Karikar C., Maitra A., Maitra A. Polymeric nanoparticle-encapsulated curcumin (“nanocurcumin”): A novel strategy for human cancer therapy. Journal of Nanobiotechnology. 2007; 5:3-20.
40. Liu Y., Fuchs J., Li C., Lin J. IL-6, a risk factor for hepatocellular carcinoma: FLLL32 inhibits IL-6 induced STAT3 phosphorylation in human hepatocellular cancer cells. Cell Cycle. 2010; 9:3423-3427
41. Wang X., Wang Q., Ives K L., Evers B M. Curcumin inhibits neurotensin-mediated interleukin-8 production and migration of HCT116 human colon cancer cells. Clin Cancer Res. 2006; 12: 5346-5355.
42. Bharti A C., Donato N., Aggarwal B B. Curcumin (diferuloylmethane) inhibits constitutive and IL-6-inducible STAT3 phosphorylation in human multiple myeloma cells. J Immunol. 2003; 171:3863-3871.
43. Park J., Ayyappan V., Bae E K., Lee C., Kim B S., Kim B K., Lee Y Y., Ahn K S., Yoon S S. Curcumin in combination with bortezomib synergistically induced apoptosis in human multiple myeloma U266 cells. Molecular Oncology. 2008; 2:317-326.
44. Krupkova O, Sekiguchi M, Klasen J, Hausmann O, Konno S, Ferguson S J, Wuertz-Kozak K. Epigallocatechin 3-gallatee surpresses interleukin-1β-induced inflammatory responses in intervertebral disc cells in vitro and reduces radiculopathic pain in rats. European Cells and Materials. 2014; 28:372-386.
45. Wuertz K, Quero L, Sekiguchi M, Klawitter M, Nerlich A, Konno S, Kikuchi S, Boos N. The red wine polyphenol resveratrol shows promising potential for the treatment of nucleus pulposus-mediated pain in vitro and in vivo. Spine. 2011; 36:E1373-E1384.
46. Genevay S, Finckh A, Mezin F, Tessitore E and Guerne P A: Influence of cytokine inhibitors on concentration and activity of MMP-1 and MMP-3 in disc herniation. Arthritis Research and Therapy. 2009; 11:R169-R176.
47. Shakibaei M, John T, Schulze-Tanzil G, Lehmann I, Mobasheri A. Suppression of NF-κB activation by curcumin leads to inhibition of expression of cyclo-oxygenase-2 and matrix metalloproteinase-9 in human articular chondrocytes: Implications for the treatment of osteoarthritis. Biochem Pharmacol. 2007; 73:1434-1445.
48. Clutterbuck A L, Allaway D, Harris P, Mobasheri A. Curcumin reduces prostaglandin E2, matrix metalloproteinase-3 and proteoglycan release in the secretome of interleukin 1β-treated articular cartilage. F1000 Research. 2013; 2:147-162.
49. Schulze-Tanzil G, Mobasheri A, Sendzik J, John T and Shakibaei M. Effects of curcumin (diferuloylmethane) on nuclear factor kappaB signaling in interleukin-1beta-stimulated chondrocytes. Ann NY Acad Sci. 2004; 1030:578-586.
50. Csaki C, Mobasheri A and Shakibaei M. Synergistic chondroprotective effects of curcumin and resveratrol in human articular chondrocytes: Inhibition of IL-1β induced NF-κB mediated inflammation and apoptosis. Arthritis Research and Therapy. 2009; 11:R165-R182.
51. Liacini A, Sylvester J, Li W Q, Huang W, Dehnade F, Ahmad M and Zafarullah M. Induction of matrix metalloproteinase-13 gene expression by TNF-alpha is mediated by MAP kinases, AP-1, and NF-kappaB transcription factors in articular chondrocytes. Exp Cell Res. 2003; 288:208-217.
52. Yu Z, Xu N, Wang W, Pan S, Li K, Liu J. Interleukin-1 inhibits SOX9 and collagen type II expression via nuclear factor-KB in the cultured human intervertebral disc cells. Chinese Medical Journal. 2009; 122:2483-2488.

53. Klawitter M, Quero L, Klasen J, Gloess A N, Klopprogge B, Hausmann O, Boos N, Wuertz K. Curcumin DMSO extracts and curcumin exhibit an anti-inflammatory and anti-catabolic effect on human intervertebral disc cells, possibly influencing TLR2 expression and JNK activity. Journal of Inflammation. 2012; 9:29-33.
54. Di Pierro F and Settembre R: Safety and efficacy of an add-on therapy with curcumin phytosome and piperine and/or lipoic acid in subjects with a diagnosis of peripheral neuropathy treated with dexibuprofen. Journal of Pain Research. 2013; 6:497-503.
55. Weiler C, Nerlich A G, Zipperer J, Bachmeier B E and Boos N. 2002 SSE Award competition in basic science: Expression of major matrix metalloproteinases is associated with intervertebral disc degradation and resorption. Eur Spine J. 2002; 11: 308-320.
56. LeMaitre C L, Freemont A J and Hoyland J A. Localization of degratative enzymes and their inhibitors in the degenerate human intervertebral disc. Journal of Pathology. 2004; 204: 47-54.
57. LeMaitre C L, Freemont A J and Hoyland J A. Human disc degeneration is associated with increased MMP-7 expression. Biotech Histochem. 2006; 81:125-131.
58. Richardson S M, Doyle P, Minogue B M, Gnanalingham K and Hoylan J A. Increased expression of matrix metalloproteinase-10, nerve growth factor and substance P in the painful degenerative intervertebral disc. Arthritis Research and Therapy. 2009; 11: R126-R146.
59. Gruber H E, Ingram J A, Hoelscher G L, Zinchenko N, Norton H J and Hnaley E N. Matrix metalloproteinase 28, a novel matrix metalloproteinase, is constitutively expressed in human intervertebral disc tissue and is present in matrix of more degenerated discs. Arthritis Research and Therapy. 2009; 11: R184.
60. Wan Z Y, Sun Z, Song F, Chen Y F, Zhang W L, Wang H Q and Luo Z J: Downregulated interleukin 37 expression associated with aggravation of intervertebral disc degeneration. Int J Clin Exp Pathol. 2014; 7: 656-662.
61. Akyol S, Senel-Eraslan B, Etyemez H, Tanriverdi T and Hanci M: Catabolic cytokine expressions in patients with degenerative disc disease. Turkish Neurosurgery. 2010; 20:492-499.
62. Ahn S H, Cho Y W, Ahn M W, Jang S H, Sohn Y K and Kim H S: mRNA expression of cytokines and chemokines in herniated lumbar intervertebral discs. Spine. 2002; 27:911-917.
63. Gruber H E, Hoelscher G L, Ingram J A, Norton H J and Hanley E N: Increased IL-17 expression in degenerated human discs and increased production in cultured annulus cells exposed to IL-1β and TNF-α. Biotech Histochem. 2013; 88:302-310.
64. Jang S, Chun J, Shin E M, Kim H, Kim Y S. Inhibitory effects of curcuminoids from Curcuma longa on matrix metalloproteinase-1 expression in keratinocytes and fibroblasts. Journal of Pharmaceutical Investigation. 2012; doi.10.1007/s40005-012-0005-8.Zhon
65. Zhong, Yu, Feng, Li. Curcumin suppresses tumor necrosis factor α induced matrix metalloproteinase 2 expression and activity in rat vascular smooth muscle cells via the NFκB pathway. Experimental and Therapeutic Medicine. 2014; 7:1653-1658.
66. Zhang Y. Design synthesis and biological evaluation of novel curcumin analogues as inhibitors of matrix metalloproteinases and pro-inflammatory cytokines. Ph.D. Dissertation State University of New York at Stony Brook. 2012; 298:3551749.
67. Yodkeeree S, Garbisa S, Limtrakul P. Tetrahydrocurcumin inhibits HT1080 cell migration and invasion via downregulation of MMPs and uPA. Acta Pharmacologica Sinica. 2008; 29:853-860.
68. Su C C, Chen G W, Lin J G, Wu L T, Chung J G. Curcumin inhibits cell migration of human colon cancer Colo 205 cells through the inhibition of nuclear factor kappa B/p65 and down-regulates cyclooxygenase-2 and matrix metalloproteinase-2 expression. Anticancer Research. 2006; 26:1281-1288.
69. Chung C C, Kao Y H, Liou J P, Chen Y J. Curcumin surpress cardiac fibroblasts activities by regulating proliferation, migration, and the extracellular matrix. Acta Cardiol Sin. 2014; 30:474-482.
70. Elburki M S, Rossa C, Guimaraes M R, Goodenough M, Lee H M, Curylofo F A, Zhang Y, Johnson F, Golub L M. A novel chemically modified curcumin reduces severity of experimental periodontal disease in rats: Initial observations. Mediators of Inflammation. 2014; http://dx.doi.org/10.1155/2014/959471.
71. Swarnakar S, Paul S. Curcumin arrests endometriosis by downregulation of matrix metalloproteinase-9 activity. Indian J Biochem Biophys. 2009; 46:59-65.
72. Cao J, Han Z, Tian L, Chen K, Fan Y, Ye B, Huang W, Wang C, Huang Z. Curcumin inhibits EMMPRIN and MMP-9 expression through AMPK-MAPK and PKC signaling in PMA induced macrophages. Journal of Translational Medicine. 2014; 12:266-275.
73. Lee K W, Kim J H, Lee H J, Surh Y J. Curcumin inhibits phorbol ester-induced up-regulation of cyclooxygenase-2 and matrix metalloproteinase-9 by blocking ERK1/2 phosphorylation and NF-κB transcriptional activity in MCF10A human breast epithelial cells. Antioxidants and Redox Signaling. 2005; 7:1612-1620.
74. Shen F, Cai W S, Li J L, Feng Z, Liu Q C, Xiao H Q, Cao J, Xu B. Synergism from the combination of ulinastatin and curcumin offers greater inhibition against colorectal cancer liver metastases via modulating matrix metalloproteinase-9 and E-cadherin expression. OncoTargets and Therapy. 2013; 6:523-526.
75. Xu Y, Hu B, Anders R, Maitra A, Fan J. Use of nanocurcumin to inhibit proliferation and metastases of hepatocellular carcinoma via NF-κB mediated matrix metalloproteinase-9 downregulation. J Clin Oncol. 2013; 31:e22103.
76. Tsang R K, Tang W W, Gao W, Ho W K, Chan J Y, Wei W I and Wong T S. Curcumin inhibits tongue carcinoma cells migration and invasion through downregulation of matrix metallopeptidase 10. Cancer Investigation. 2012; 30:503-512.
77. Xu Y X, Pindolia K R, Janakiraman N, Chapman R A and Gautam S C: Curcumin inhibits IL-1α and TNF-α induction of AP-1 and NF-κB DNA-binding activity in bone marrow stromal cells. Hematopathol Mol Hematol. 1997-1998; 11:49-62.
78. Jobin C, Bradham C A, Russo M P, Juma B, Narula A S, Brenner D A and Sartor R B: Curcumin blocks cytokine-mediated NF-κB activation and proinflammatory gene expression by inhibiting inhibitory factor I-κB kinase activity. J Immunol. 1999; 163:3474-3483.
79. Henrotin Y, Clutterbuck A L, Allaway D, Lodwig E M, Harris P, Mathy-Hartert M, Shakibaei M and Mobasheri A: Biological actions of curcumin on articular chondrocytes. Osteoarthritis Cartilage. 2010; 18:141-149.
80. Ganjali S, Sahebkar A, Mandipour E, Jamialahmadi K, Torabi S, Akhlaghi S, Ferns G, Parizadeh S M R and Ghayour-Mobarhan M: Investigation of the effects of curcumin on serum cytokines in obese individuals: A randomized controlled study. Sci World J http://dx.doi.org/10.1155/2014/898361.
81. Wang W, Zhu R, Xie Q, Li A, Xaio Y, Li K, Liu H, Cui D, Chen Y and Wang S: Enhanced bioavailability and efficiency of curcumin for the treatment of asthma by its formulation in solid lipid nanoparticles. Int J Nanomed. 2012; 7:3667-3677.
82. Biswas S K, McClure D, Jimenez L A, Megson I L and Rahman I: Curcumin induces glutathione biosynthesis and inhibits NF-κB activation and interleukin-8 release in alveolar epithelial cells: Mechanism of free radical scavenging activity. Antioxid Redox Signal. 2005; 7:32-41.
83. Fahey A J, Robins R A and Constantinescu C S: Curcumin modulation of IFN-β and IL-12 signaling and cytokine induction in human T cells. J Cell Mol Med. 2007; 11:1129-1137.
84. Okamoto Y, Tanaka M, Fukui T and Masuzawa T: Inhibition of interleukin 17 production by curcumin in mice with collagen-induced arthritis. Biomed Res. 2011; 22:299-304.

What is claimed is:

1. A method of treating an intervertebral disc disease or condition comprising:
identifying a patient in need of treatment for the intervertebral disc disease or condition; and
administering to the patient an amount of an anti-inflammatory agent that causes a cardiac channelopathy or cardiotoxicity and a liposome, wherein the liposome is provided in an amount sufficient to reduce or eliminate the cardiac channelopathy caused by the anti-inflammatory agent, and the anti-inflammatory agent is provided in an amount sufficient to treat or ameliorate the symptoms of the intervertebral disc disease or condition.

2. The method of claim 1, wherein the liposomes are defined further as empty liposomes and are provided in an amount sufficient to reduce or eliminate the QT prolongation.

3. The method of claim 1, wherein the anti-inflammatory agent that causes a cardiac channelopathy is a curcumin or a curcuminoid.

4. The method of claim 1, wherein the liposome comprises at least one of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, or diacylglycerolsuccinate.

5. The method of claim 3, wherein the therapeutically effective amount comprises 50 nM/kg, 10 to 100 nM/kg, 25 to 75 nM/kg, 10, 20, 30, 40, 50, 60, 70, 80, 90, or 100 nM/kg curcumin or curcuminoids of body weight of the subject.

6. The method of claim 3, wherein the curcumin is a synthetic curcumin and is 85, 86, 87, 88, 89, 90, 91, 92, 93, 94, 95 or 96% pure diferuloylmethane.

7. The method of claim 3, wherein the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.

8. The method of claim 3, wherein the curcumin or curcuminoid and the liposomes are adapted to be delivered enterally, parenterally, intravenously, intraperitoneally, or orally.

9. The method of claim 3, wherein the curcumin or curcuminoid and the liposomes are adapted to be injected intervertebrally.

10. The method of claim 3, wherein the curcumin or curcuminoid and the liposomes further reduce or eliminate pain caused by the intervertebral disease or condition.

11. The method of claim 1, wherein the anti-inflammatory agent is selected from at least one of celecoxib; sulindac; oxaprozin; salsalate; diflunisal; piroxicam; indomethacin; etodolac; meloxicam; naproxen; nabumetone; ketorolac tromethamine; naproxen/esomeprazole; serrapeptase; or diclofenac, in an amount that causes a cardiopathy or cardiotoxicity.

12. A method of reducing cytokine release and inflammation caused by an intervertebral disc disease or condition comprising:
identifying a patient in need of treatment for the intervertebral disc disease or condition; and
administering to the patient an amount of a curcumin or a curcuminoid and a liposome sufficient to reduce or ameliorate the cytokine release and inflammation caused by the intervertebral disc disease or condition.

13. The method of claim 12, wherein the liposomes are defined further as empty liposomes and are provided in an amount sufficient to reduce or eliminate the QT prolongation caused by the curcumin or the curcuminoids.

14. The method of claim 12, wherein the liposome comprises at least one of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, or diacylglycerolsuccinate.

15. The method of claim 12, wherein the therapeutically effective amount comprises 50 nM/kg, 10 to 100 nM/kg, 25 to 75 nM/kg, 10, 20, 30, 40, 50, 60, 70, 80, 90, or 100 nM/kg curcumin or curcuminoids of body weight of the subject.

16. The method of claim 12, wherein the synthetic curcumin is 85, 86, 87, 88, 89, 90, 91, 92, 93, 94, 95 or 96% pure diferuloylmethane.

17. The method of claim 12, wherein the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.

18. The method of claim 12, wherein the curcumin or curcuminoid and the liposomes are adapted to be delivered enterally, parenterally, intravenously, intraperitoneally, or orally.

19. The method of claim 12, wherein the curcumin or curcuminoid and the liposomes are adapted to be injected intervertebrally.

20. The method of claim 12, wherein the curcumin or curcuminoid and the liposomes further reduce or eliminate pain caused by the intervertebral disease or condition.

21. A method of determining if a candidate drug causes an amelioration symptoms or treats one or more adverse reactions triggered by an intervertebral disc disease or condition in a subject, the method comprising:
(a) administering an amount of an anti-inflammatory agent that causes a cardiac channelopathy or cardiotoxicity in combination with empty liposomes, and a placebo to a second subset of the patients, wherein the candidate drug is provided in an amount effective to reduce or prevent the overall level of intervertebral cytokines in the subject;
(b) measuring the level of cytokines in the subject from the first and second set of patients; and
(c) determining if the anti-inflammatory agent in combination with empty liposomes ameliorates symptoms or treats one or more adverse reactions triggered by the intervertebral disease or condition that triggers a intervertebral cytokines that is statistically significant as compared to any reduction occurring in the subset of patients that took the placebo, wherein a statistically significant reduction indicates that the candidate drug is useful in treating the intervertebral disc disease or condition while also reducing or eliminating the overall level of the intervertebral cytokines.

22. The method of claim 21, wherein the anti-inflammatory agent is selected from at least one of curcumin; celecoxib; sulindac; oxaprozin; salsalate; diflunisal; piroxicam; indomethacin; etodolac; meloxicam; naproxen; nabumetone; ketorolac tromethamine; naproxen/esomeprazole; serrapeptase; or diclofenac, in an amount that causes a cardiopathy or cardiotoxicity.
